(19) United States
(12) Patent Application Publication
(10) Pub. No.: US 2009/0148498 A1
(43) Pub. Date: Jun. 11, 2009

(54) Controlled release implantable dispensing device and method

(75) Inventors: Barry M. Libin, Great Neck, NY (US); Jeffrey M. Liebmann, Great Neck, NY (US); Weiliam Chen, Mt. Sinai, NY (US)

Correspondence Address: HEDMAN & COSTIGAN P.C., 1185 AVENUE OF THE AMERICAS, NEW YORK, NY, 10036, US

(73) Assignee: SUSTAINED NANO SYSTEMS LLC, Great Neck, NY (US)

(21) Appl. No.: 12/291,841

(22) Filed: Nov. 13, 2008

Related U.S. Application Data
(63) Continuation-in-part of application No. 12/152,459, filed on May 14, 2008.
(60) Provisional application No. 60/930,105, filed on May 14, 2007.

(51) Int. Cl. A61F 2/14; A61K 9/14; A61K 31/56

(52) U.S. Cl. 424/427; 424/501; 514/183

(57) ABSTRACT

A dispensing device having a polymer which is combined with a therapeutic agent in the form of a microparticle which is compressed to form a controlled release dispensing device and methods of locally administering a therapeutic agent using said microparticles.

### CROSS REFERENCE TO RELATED APPLICATIONS

This application is a continuation in part of Ser. No. 12/152,459, filed May 14, 2008.

### FIELD OF THE INVENTION

This invention relates to the field of controlled release implantable drug delivery devices.

### BACKGROUND OF THE INVENTION

One of the major issues involving treatment involves the toxicity and/or adverse effects of pharmaceuticals that complicate the treatment of various conditions. Systemically administered medications tend to have effects that are undesirable when the therapeutic objective of the treatment is considered. If a pathology affects only a particular part or organ in the body, it is desirable to only administer treatment to that particular part or organ. In the prior art it has been known to provide localized radiation treatment by implanting radioactive pharmaceuticals in an organ that is to be treated so that radiation will be substantially confined to that organ. Most other implants have been intended to provide a systemic effect.

Two sustained delivery systems in the form of ophthalmic inserts that have been developed for commercial use are the Ocusert system (Akorn) and Lacrisert® (Aton). The Ocusert device is designed to provide for the release of medication at predetermined and predictable rates, which permits the elimination of frequent dosing by the patient, ensures nighttime medication, and provides a better means of patient compliance. The insert is elliptical with dimensions of 13.4 by 4.7 mm and 0.3 mm in thickness. The insert is flexible and is a multilayered structure consisting of a drug-containing core surrounded on each side by a layer of copolymer membranes through which the drug diffuses at a constant rate. The rate of drug diffusion is controlled by the polymer composition, the membrane thickness, and the solubility of the drug. The devices are sterile and do not contain preservatives. Ocusert inserts containing pilocarpine have been used in glaucoma therapy. After placement in the conjunctival fornix, the inserts are designed to release medication at the desired rates over a 7-day period at which time they are removed and replaced with new ones.

The Lacrisert® insert is a sterile, translucent, rod-shaped, water-soluble form of hydroxypropyl cellulose. The product is inserted into the inferior cul-de-sac of the eye of patients with dry eye states. The insert acts to stabilize and thicken the precorneal tear film and to delay its breakup. Inserts are typically placed in the eye once or twice daily. Following administration, the inserts soften and slowly dissolve.

The following U.S. patents disclose various ocular inserts for medicinal therapy. U.S. Pat. No. 4,730,013 to J. V. Bondi, et al., assigned to Merck & Company, Inc., discloses ocular inserts with or without pharmaceutically active agents, comprising 75% to 100% of a matrix of 15% polyvinyl alcohol, 10% glycerine, 75% hydroxy propyl methylcellulose phthalate, and 0-25% of a pharmacologically active agent. U.S. Pat. No. 5,637,085 describes the making of an implantable wafer for the treatment of solid cancer tumors.

U.S. Pat. No. 4,522,829 to Andreas Fuchs, et al., (Merck GmbH), discloses an intraocular pressure-lowering film insert of a 1-(p-2-iso-propoxyethoxy methyl-phenoxy)-3-isopropylamino-propan-2-ol or a physiologically acceptable salt thereof and an ophthalmically acceptable carrier.

U.S. Pat. No. 4,432,964 to Robert M. Gale (Alza Corp.) discloses an ocular insert for treating inflammation made of a pair of micronized steroids consisting of two topically acceptable different chemical therapeutic forms of betamethasone or a derivative, and a bio-erodible polymeric polyorthoester carrier.

U.S. Pat. No. 4,346,709 to Edward E. Schmitt (Alza Corp.) discloses an erodible device for delivering a drug to an environment of use, which includes a poly(orthoester) or a poly(orthocarbonate).

U.S. Pat. No. 4,303,637 to Robert M. Gale, et al., discloses an ocular insert composed of a beta blocking drug in a polymer with the drug surrounded by the polymer selected from the group consisting of poly(olefin), poly(vinylolefin), poly(haloolefin), poly(styrene), poly(vinyl), poly(acrylate), poly(methacrylate), poly(oxide), poly(ester), poly(amide), and poly(carbonate).

U.S. Pat. No. 4,190,642 (Alza Corp.) discloses an ocular insert composed of a discrete depot of a pilocarpine solute and an epinephrine solute, a film of an ethylene-vinyl ester copolymer forming the insert, where fluid from the environment is imbibed through the wall into the depots to continually dissolve the solutes and release the formulation.

U.S. Pat. No. 4,093,709 to Nam S. Choi (Alza Corp.) discloses an ocular insert composed of an orthoester and an orthocarbonate polymer.

U.S. Pat. No. 3,993,071, issued Nov. 23, 1976 to Takeru Higuchi, et al., discloses a bio-erodible ocular insert for the controlled administration of a drug to the eye from 8 hours to 30 days, in which the drug formulation can also be microencapsulated and the microcapsules dispersed in the drug release rate controlling material.

U.S. Pat. No. 3,981,303 to Takeru Higuchi, et al. (Alza Corp.) discloses an ocular insert for the continuous controlled administration of a drug to the eye composed of a plurality of microcapsule reservoirs comprised of a drug formulation confined within a drug release rate controlling material, distributed throughout a bio-erodible matrix permeable to the passage of the drug at a higher rate than the rate of drug passage through the drug release rate controlling material, where the device is of an initial shape and size that is adapted for insertion and retention in the sac of the eye. The hydrophobic material may be selected from cholesterol, waxes, C.sub.10 to C.sub.20 fatty acids, and polyesters, and the drug may be selected from epinephrine, pilocarpine, hydrocortisone, idoxuridine, tetracycline, polymixin, gentamycin, neomycin, and dexamethasone.

U.S. Pat. No. 3,960,150 to Takeru Higuchi, et al. (Alza Corp.) discloses an ocular insert for the continuous controlled administration of a drug to the eye composed of a body of hydrophobic bio-erodible drug release rate controlling material containing a drug, where the body is of an initial shape adapted for insertion in the sac of the eye, where the drug release rate controlling material can be a polyester, and the drug may be selected from epinephrine, pilocarpine, hydrocortisone, idoxuridine, tetracycline, polymixin, gentamycin, neomycin, and dexamethasone, and derivatives.

U.S. Pat. No. 3,811,444, issued May 21, 1974 to Richard W. Baker, et al., assigned to the Alza Corp., discloses an ocular insert for the continuous controlled administration of a drug to the eye comprising a drug formulation dispersed through a body of selected hydrophobic polycarboxylic acid which erodes over time to dispense the desired amount of drug. The polycarboxylic acid can be a copolymer of an acid from the group of maleic acid, acrylic acid, lower alkyl acrylic acids from about 4 to about 6 carbon atoms, with a copolymerizable olefinically unsaturated material selected from the group consisting of ethylene, propylene, butadiene, isoprene and styrene and the lower alkyl vinyl ethers.

U.S. Pat. No. 3,630,200, issued Dec. 28, 1971, to Takeru Higuchi, assigned to the Alza Corporation, discloses a drug-dispensing ocular insert for insertion into the cul-de-sac of the conjunctiva between the sclera of the eyeball and the lid where the inner core contains the drug and is surrounded by a soft hydrophilic outer layer, where the outer layer can be composed of a polymer selected from the group consisting of hydrophilic hydrogel of an ester of acrylic or methacrylic acid, modified collagen, cross-linked hydrophilic polyether gel, cross-linked polyvinyl alcohol, and cross-linked partially hydrolyzed polyvinyl acetate and cellulosic gel. The inner core may be a polymer selected from the group of plasticized or unplasticized polyvinylchloride, plasticized nylon, unplasticized soft nylon, silicone rubber, polyethylene, hydrophilic hydrogel of an ester of acrylic or methacrylic acid, modified collagen, cross-linked hydrophilic polyether gel, cross-linked polyvinyl alcohol, cross-linked partially-hydrolyzed polyvinylacetate, cellulosic gel, ion-exchange resin and plasticized polyethylene terephthalate.

U.S. Pat. No. 3,618,604 to Richard A. Mess (Alza Corporation) discloses a drug-dispensing ocular insert adapted for insertion into the cul-de-sac of the eye, where the insert is a tablet containing a reservoir of drug formulation within a flexible polymeric material, and the polymeric material is formed of plasticized or unplasticized polyvinylchloride, plasticized nylon, unplasticized soft nylon, plasticized polyethylene terephthalate, silicon rubber, hydrophilic hydrogel of a ester of acrylic or methacrylic acid, modified collagen, cross-linked hydrophilic polyether gel, cross-linked polyvinyl alcohol, and cross-linked partially-hydrolyzed polyvinylacetate.

U.S. Pat. Nos. 3,993,071; 3,986,510; 3,981,303, 3,960,150, and 3,995,635 to Higuchi, et al., disclose a biodegradable ocular insert made from zinc alginate, poly(lactic acid), poly(vinyl alcohol), poly(anhydrides), and poly(glycolic acid).

A number of patents disclose the use of drug-loaded polyanhydrides (wherein the anhydride is in the backbone of the polymer) as matrix materials for ocular inserts. See, in general, U.S. Pat. Nos. 5,270,419; 5,240,963; and 5,137,728. Other U.S. patents that describe the use of polyanhydrides for controlled delivery of substances include: U.S. Pat. No. 4,857,311 to Domb and Langer, entitled “Polyanhydrides with Improved Hydrolytic Degradation Properties,” which describes polyanhydrides with a uniform distribution of aliphatic and aromatic residues in the chain, prepared by polymerizing a dicarboxylic acid with an aromatic end and an aliphatic end); U.S. Pat. No. 4,888,176 to Langer, Domb, Laurencin, and Mathiowitz, entitled “Controlled Drug Delivery High Molecular Weight Polyanhydrides,” which describes the preparation of high molecular weight polyanhydrides in combination with bioactive compounds for use in controlled delivery devices); and U.S. Pat. No. 4,789,724 to Domb and Langer, entitled “Preparation of Anhydride Copolymers,” which describes the preparation of very pure anhydride copolymers of aromatic and aliphatic diacids.

U.S. Pat. No. 5,075,104 discloses an ophthalmic carboxyvinyl polymer gel for the treatment of dry eye syndrome.

U.S. Pat. No. 4,407,792 discloses an aqueous gel that includes a gel-forming amount of an ethylene-maleic anhydride polymer.

U.S. Pat. No. 4,248,855 discloses the salt of pilocarpine with a polymer containing acid groups for use as an ocular insert, among other things.

U.S. Pat. No. 4,180,064 and U.S. Pat. No. 4,014,987 disclose the use of poly(carboxylic acids) or their partially esterified derivatives as drug delivery devices.

PCT/US90/07652 discloses that biologically active compounds containing a carboxylic acid group can be delivered in the form of an anhydride of a carrier molecule that modifies the properties of the molecule. U.S. Pat. No. 5,322,691 discloses the use of pressure to form drug containing ocular inserts from polymers with pressures up to 12 tons. The insets are made by mixing the drug powder with a polymer prior to compressing the mixture. There is no mention of the application of pressure to microspheres and polymers to form \a dispensing device.

Although these patents disclose a number of types of ocular inserts, there is still a need to provide new dosage forms with modified properties for the delivery of local delivery of therapeutic agents. In particular, there is a need to provide a dispensing device that provides for the long acting local delivery of therapeutic agents to the eye and other locations in the body. The formulations comprise a matrix of a polymer carrier and an active drug where the matrix is made by compression of micro or nano particles of a therapeutic agent in combination with a polymer. The matrix is positioned in or near the location where it will make available the therapeutic agent for treating pathologic conditions. The preferred polymeric matrix combines the characteristics of stability, strength, flexibility, low melting point, dispersability and suitable degradation profile. The matrix must retain its integrity for a suitable time so that it may be handled and placed in an aqueous environment, such as the eye, pancreas, liver, adrenal gland, colon, without loss of structural integrity. It should also be stable enough to be stored an shipped without loss of structural integrity. The matrix is designed to disintegrate into its constituent particles shortly after it is placed in position to release the therapeutic agent.

Gliadel Implant Wafer Generic Name: Carmustine in Polifeprosan Intracranial Implant Wafer (kar-MUS-teen/poh-LIF-eh-pro-sin) Brand Name: Gliadel Gliadel is a white, dime-sized wafer made up of a biocompatible polymer that contains the cancer chemotherapeutic drug, carmustine (BCNU). After a neurosurgeon removes a high-grade malignant glioma, up to eight wafers can be implanted in the cavity where the tumor resided. Once implanted, Gliadel slowly dissolves, releasing high concentrations of BCNU into the tumor site. The specificity of Gliadel minimizes drug exposure to other areas of the body.

There is a need to provide a dispensing device that provides for the local delivery of long acting formulations of therapeutic agents. The applicants have devised formulations which comprise a matrix of a polymer carrier and an active drug where the matrix is made by (hyper?)compression of micro or nano particles of a therapeutic agent in combination with a polymer. The matrix is positioned in the body in a location where it will be available for absorption to produce a substantially local effect. The preferred polymeric matrix combines the characteristics of stability, strength, flexibility, low melting point, dispersability and suitable degradation profile. The matrix must retain its integrity for a suitable time so that it may be handled and placed in an aqueous environment without loss of structural integrity. It should also be stable enough to be stored and shipped without loss of structural integrity. The matrix is designed to disintegrate into its constituent particles shortly after it is placed in position to release the therapeutic agent to the area where it will be available for therapeutic purposes.

### SUMMARY OF THE INVENTION

The invention provides a controlled release drug delivery device for local delivery of a therapeutic agent that comprises a matrix that is made by compressing units of microparticles or nanoparticles that comprise a therapeutically compatible polymer and a therapeutic agent.

The compressed unit is shaped in such a manner that the compressed unit may be implanted or injected under the skin e.g. subcutaneously or intramuscularly, or within the tissue of a specific bodily organ or structure, where it will continuously deliver a drug for local absorption.

The invention also includes a method of administering a therapeutic agent which comprises (a) forming a dosage form comprising a polymer in combination with a agent in the form of a microparticles or nanoparticles; (b) compressing the microparticles or nanoparticles to form a controlled release dispensing unit; and (c) thereafter implanting or injection said dispensing unit in a location in the body requiring localized treatment of a pathological condition with a therapeutic agent.

Accordingly, it is an object of the invention to provide a dispensing device for use as an implantable or injectable controlled release device for the local treatment of a pathological condition with a therapeutic agents over a period of time.

It is also an object of this invention to improve patient compliance with physician directed administration of therapeutic agents by minimizing the number of doses and maximizing the local effect of a therapeutic effect from a specific dose.

It is therefore an object of the present invention to provide a method for the localized treatment of pathologic conditions using a matrix that is made by compressing units of microparticles or nanoparticles that comprise a therapeutically compatible polymer and a therapeutic agent.

It is also an object of the invention to provide a dispensing device that is made by compressing microparticles or nanoparticles containing a therapeutic agent and a compressed polymer which will release a therapeutic agent over an extended period of time.

It is also an object of the invention to provide hydrophilic or preferably, hydrophobic drugs for ophthalmic pathologies or other types of pathology, drugs in this type of system. Such drugs include antibacterials, antibiotics, anti-inflammatory agents, immunosuppressive agents, antiglaucoma agents etc.

It is also an object of this invention to avoid active patient involvement with the administration of a therapeutic agent by having a physician place a dispensing device in a position where it will locally deliver the therapeutic agent over an extended period of time without any action on the part of the patient.

It is also an object of this invention to provide a dispensing device that will provide local controlled release of a therapeutic agent from a non-toxic biodegradable polymer system that does not have to be removed from the body after exhaustion of a therapeutic agent from a dispensing device.

It is also an object of the invention to provide a convenient method of handling microcapsules by forming them into a hypercompressed dosage unit.

It is also an object of the invention to provide a method of locally administering a drug which comprises forming a dispensing device comprising a polymer in combination with a therapeutic agent in the form of a microparticle which is compressed to form a controlled release dispensing unit and thereafter placing said controlled release dispensing unit in contact with an injectable liquid to disperse the microparticles and form a suspension of microparticles prior to placing said suspension in a patient in a location that will provide for release of the drug.

These and other objects of the invention will become apparent from a review of the present specification.

### DESCRIPTION OF THE SEVERAL VIEWS OF THE DRAWINGS

FIG. 1 is a photomicrograph of uncompressed microparticles according to Example 1

FIG. 2 is a photomicrograph of compressed microparticles according to Example 1

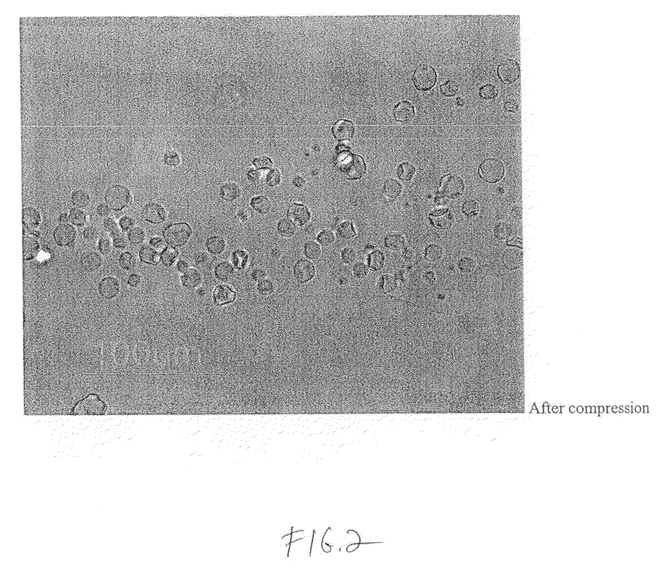

FIG. 3 is a table that reports the level of dexamethasone detected in the vitreous humor and in the aqueous humor.

FIG. 4 is a graph which shows the rate of in vitro release of dexamethasone from microspheres of the invention.

FIG. 5 is a partial cutaway diagram of a syringe that is provided for implanting microparticles that are dispersed from a dispensing device of the invention.

### DETAILED DESCRIPTION OF THE INVENTION

The dispensing device of the invention comprises a polymer that is combined with a therapeutic agent and compressed to form a controlled release dispensing unit. The therapeutic agents that may be mixed with the polymer comprise hydrophilic or preferably, hydrophobic drugs that are antifungal, antibacterial, antibiotic, anti-inflammatory, immunosuppressive, tissue growth factors, dentinal desensitizers, antioxidants, nutritional agents, vitamins, odor masking agents for example. Specific examples include steroids, non-steroidal anti-inflammatory drugs, antihistamines, antibiotics, mydriatics, beta-adrenergic antagonists, anesthetics, alpha-2-beta adrenergic agonists, mast cell stabilizers, prostaglandin analogues, sympathomimetics, parasympathomimetics, antiproliferative agents, agents to reduce angiogenesis and neovascularization, vasoconstrictors and combinations thereof and any other agents designed to treat disease, such as a anti-neoplastic agent, a polynucleotide, or a recombinant protein analog, an angiogenic inhibitor such as Endostatin, or thalidomide; 5-fluorouracil, paclitaxol, minocycline, timolol hemihydrate, rhHGH, bleomycin, ganciclovir, huperzine, tamoxifen, piroxicam, levonorgesterel, cyclosporin and the like

Other agents include but are not limited to particular steroids but include steroids such as prednisone, methylprednisolone, dexamthasone; antibiotics including neomycin, tobramycin, aminoglycosides, fluoroquinolones, polymyxin, sulfacetamide, agents such as pilocarpine, isopilocarpine, physostigmine, demecarium, ecothiphate and acetyl choline and salts thereof; mydriatics and cycloplegics including agents such as atropine, phenylephrine, hydroxyamphetamine, cyclopentolate, homatropine, scopolamine, tropicamide and salts thereof; anesthetics include, lidocaine, proparacaine, tetracaine, phenacaine, and the like; beta-blockers such as timolol, carteolol, betaxolol, nadolol, levobunolol, carbonic anhydrase inhibitors such as dorzolamide, acetozolamide, prostaglandin analogues such as latanoprost, unoprostone, bimatoprost or travoprost.

The polymer that is used in combination with the therapeutic agent is a pharmaceutically acceptable polymer that is non-toxic and non-irritating to human tissues. These polymers include monomeric and co-polymeric materials. The preferred polymers comprise a biocompatible and biodegradable polymer that may be formed into microparticles known as microspheres or microcapsules which are typically in the size range of about 0.1 to about 150 microns, preferably from about 5 to about 120 and more preferably from about 5 to about 50 microns in diameter. The term microsphere is used to describe a substantially homogeneous structure that is obtained by mixing an active drug with suitable solvents and polymers so that the finished product comprises a drug dispersed evenly in a polymer matrix which is shaped as a microsphere. Depending on the selected size range of the microparticles the term nanoparticle may be used to describe microsphere having a diameter of between 1 μm to 1 mm. Generally a particle size should be selected so that the particles may be easily measured and transferred as necessary for the purpose of placing the particle in a suitable press for the application of pressure to form the compressed dosage form. For this purpose, a preferred range of particle sizes is from 5 to 50 μm. The compressed particles are designated as the matrix which when placed in water or in contact with aqueous body fluids will cause the compressed particles to disaggregate and form into the separate particles that were compressed to form the matrix. The hypercompressed particles can be re-dispersed in a suitable aqueous vehicle for injection or implantation. Sterile normal saline or other isotonic solutions may be used for this purpose. Since the particle size of the hypercompressed individual microspheres has been reduced, substantially more drug can be delivered using the same volume of microspheres.

Nanoparticles may be formed, for example, by sonicating a solution of polylactide polymer in chloroform containing a 2% w/w solution of polyvinyl alcohol in the presence of an therapeutic agent such as an ophthalmic therapeutic agent for 10 minutes, using a ultasonicator (Misonix XL-2020 at 50-55 W power output. Thereafter, the emulsion is stirred overnight at 4° C. to evaporate the chloroform and obtain nanoparticles of the polymer and the therapeutic agent. The medicated nanoparticles can easily access the interior of a living cell and afford the unusual opportunity of enhancing local drug therapy.

Microcapsules may also be used to form the compressed dosage forms of the invention. The term microcapsule is used to describe a dosage form, which is preferably spherical and has a polymer shell disposed around a core that contains the active drug and any added excipient which is in the size range set forth above. Generally microcapsules may be made by using one of the following techniques:

(1) phase separation methods including aqueous and organic phase separation processes, melt dispersion and spray drying;
(2) interfacial reactions including interfacial polymerization, in situ polymerization and chemical vapor depositions;
(3) physical methods, including fluidized bed spray coating; electrostatic coating and physical vapor deposition; and
(4) solvent evaporation methods or using emulsions with an anti-solvent.

In general, the microparticles are comprised of from about 0.00001 to about 50 parts by weight of therapeutic agent and is further comprised of from about 50 to about 99.99999 parts by weight of polymer per 100 parts by weight of the total weight of therapeutic agent and polymer. The preferred ranges are from 1 to 50, 5 to 40, and 20 to 30 parts by weight of therapeutic agent, the balance comprised of polymer. If desired, from 1 to 5 wt % of a binder, such as polyvinyl pyrrolidone, may be homogeneously mixed with the microparticles prior to the compression step.

The amount of drug that is implanted may vary but generally from 0.5-20% of the usual oral or intravenous dose of the drug may be employed but may vary substantially depending on the solubility, the area of implantation, the patient and the condition to be treated.

Microspheres may be formed by a typical in-emulsion-solvent-evaporation technique as described herein.

In order to provide a biodegradable polymeric matrix for a controlled release dosage form which is suitable for placement in a position where a therapeutic agent may be released for treatment of a pathology, it is preferable to select the polymer from poly(alpha hydroxy butyric acid), poly(p-dioxanone) poly(l-lactide), poly(dl-lactide), polyglycolide, poly(glycolide-co-lactide), poly(glycolide-co-dl-lactide), a block polymer of polyglycolide, trimethylene carbonate and polyethylene oxide, or a mixture of any of the foregoing. The lactide/glycolide polymers are bulk-eroding polymers (not surface eroding polymers) and the polymer will hydrolyze when formed into a microparticle matrix as water enters the matrix and the polymer decreases in molecular weight. It is possible to shift the resorption curves to longer times by increasing the polymer molecular weight, using L-polymers and decreasing the surface area by increasing the size of the microparticles or the size of the dosage form. The lactide/glycolide copolymers are available with inherent viscosities as high as 6.5 dl/g and as low as 0.15 dl/g. The lower molecular weight copolymers are preferred for the present invention. It has been found that a mol ratio of 50:50 of glycolide to lactide results in the most rapid degradation and the corresponding release of drug. By increasing the ratio of lactide in the polymer backbone from about 50 mole % to 100% the rate of release can be reduced to provide an extended therapeutic effect from a single dosage unit.

A preferred encapsulating polymer is poly(glycolide-co-dl-lactide), which serves as the preferred controlled release delivery system for the dispensing device is similar in structure to the absorbable polyglycolic acid and polyglycolic/polylactic acid suture materials. The polymeric carrier serves as a sustained-release delivery system for the therapeutic agents. The polymers undergo biodegradation through a process whereby their ester bonds are hydrolyzed to form normal metabolic compounds, lactic acid and glycolic acid and allow for release of the therapeutic agent.

Copolymers consisting of various ratios of lactic and glycolic acids have been studied for differences in rates of degradation. It is known that the biodegradation rate depends on the ratio of lactic acid to glycolic acid in the copolymer, and the 50:50 copolymer degrades most rapidly. The selection of a biodegradable polymer system avoids the necessity of removing an exhausted non-biodegradable structure from the eye with the accompanying trauma.

After the microspheres are prepared, they are compressed to form the dispensing device of the invention. The compression may be carried out in any suitable apparatus that permits the application of from 12,000 to 200,000 psi of pressure to microcapsules, and more preferably from 25,000 to 100,000 psi. and especially 50,000 to 60,000 psi The compressed dispensing device may be a flat disc, rod, pellet with rounded or smooth edges that is small enough to be placed under the skin in a location such as bones and their joints, including the knuckles, toes, knees, hips and shoulders; glands, e.g. pituitary, thyroid, prostate, ovary or pancreas, or organs, e.g. liver, brain, heart, and kidney. More particularly, the dispensing device of the invention may be utilized to treat pathology by implanting the device at or near the site of the pathology, or in a way that will affect the pathology, such as any part that comprises the body of a human or animal or fish or other living species. Such parts may include the contents of a cell, any part of the head, neck, back, thorax, abdomen, perineum, upper or lower extremities. Any part of the osteology including but not limited to the vertebral column, the skull, the thorax, including the sternum or ribs, the facial bones, the bones of the upper extremity, such as the clavicle, scapula or humerus; the bones of the hand, such as the carpus; the bones of the lower extremity, such as the ilium or the femur; the foot, such as the tarsus; joints or ligaments; muscles and fasciae; the cardiovascular system, such as the heart, the arteries, the veins, or the capillaries or blood; the lymphatic system, such as the thoracic duct, thymus or spleen; the central or peripheral nervous system, the sensory organs, such as eye, ear, nose; the skin; the respiratory system, such as the lungs, the larynx, the trachea and bronchi; the digestive system, such as the esophagus, the stomach or the liver; the urogenital system, such as the urinary bladder, the prostate, or the ovary; the endocrine glands, such as the thyroid, the parathyroid or the adrenals.

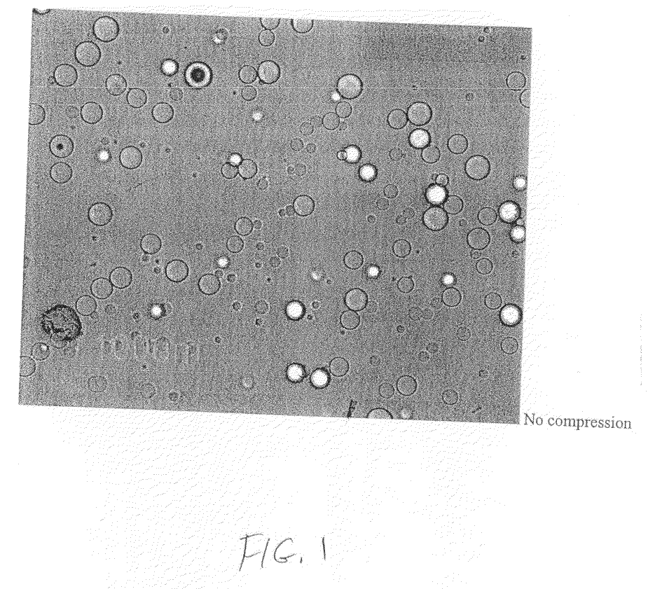

It is contemplated that the insertion of the dispensing device according to the invention will be carried out by a, such as a physician, dentist, veterinarian, nurse, or other trained professional, as it is contemplated that the method of insertion may involve procedures well known to a trained professional in order that the device will be properly placed. The dispensing device may be implanted by use of a modified syringe that will have a barrel provide with a plunger element that will extrude the dispensing device of the invention Such a device is shown in U.S. Pat. No. 5,236,355 and FIG. 1 of that patent is incorporated by reference into the present application.

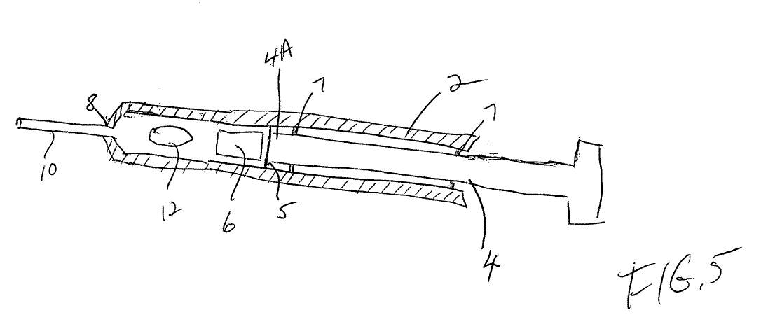

An alternative method uses a syringe, according to FIG. 5 of the present application, that is fitted with a barrel 2 and an ejector 4 which is positioned in barrel 2 by guides 7. The lower end 4A of ejector 4 is adjacent to a sterile frangible vial of an injectable liquid 6. A seal 5 is provided in the barrel 2 at the lower end of ejector 4 to prevent backflow of any liquid when the ejector is depressed to contact a frangible sterile container 6 which when broken by the action of ejector element 4 allows an injectable liquid such as water for injection, normal saline, ringers solution etc. to contact the dispensing device 12 and disperse it into microparticles so that when additional pressure is placed on the main ejector 4 in the main barrel 2 the dispersed microspheres are extruded from the wide gauge needle 10 that is mounted on barrel 2.

Generally, the thickness of the dispensing device should be from about 0.25 to 2 mm whether in the form of a disc, rod or pellet. The dispensing device in the form of e.g. a disk, should have an area equal to a circle having a diameter of about 3 to 10 mm although smaller or larger devices may be made according to the invention. A rod or cylinder shaped dosage form may be sized to be approximately 1 mm in diameter by 3 mm in length The density of the dispensing device increases as the amount of compression force is increased. The density should be sufficiently high that it reduces the rate of release of a compressed sample that is compressed using pressures of 12,000 to 200,000 psi as compared to an uncompressed sample. The compression step also allows for packing more particles into a finite volume thereby increasing drug loading and will influence the rate of drug release due to the increased density of the compressed dosage form. The invention also includes dispensing devices which have two or more drugs formed into microparticles or nanoparticles with a polymer in order to provide controlled release of drugs intended for combination therapy.

Where complete surgical removal of a neoplasm is not possible, the implantation of the hypercompressed delivery device as such or in dispersed form may be applied, wherever the neoplasm is located, will allow for the continuous release of drug, such as 5-fluorouracil, or taxol. The implantation may take place with or without surgical intervention, or it may be implanted or positioned in the course of a surgical procedure where it is not possible to completely remove all affected tissues using an appropriate injector as described herein. The implantation of the hypercompressed particles of the invention will reduce or avoid the severe systemic side effects of chemotherapy which may cause serious side effects, including damage to healthy skin, and mucosa lining the oral, pharyngeal, esophageal and gastrointestinal tracts. For example, the severe, dose-limiting, painfully debilitating side effect of oral and gastromucositis, resulting from direct contact of the drug when taken orally, or from intravenous administration will be reduced or eliminated. The dose of the drug will depend on the size and location of the neoplasm but generally the implanted dose will be from 0.5-5% or more preferably 1-2% of the systemic dose and will depend on the response of particular neoplasms, the age and condition of the patient, the nature of the pathology as well as any prior therapy. In the case of carmustine which is used alone or in combination with other anti-cancer drugs for local implantation for the treatment of glial tumors, a dose of 5-10 mg may be used by implantation once every 2 to 4 weeks and 5-fluorouracil may be used for pancreatic cancers by the implantation every 2 to 4 weeks of a dispensing device in the affected area which has from 1-2 mg of 5-fluorouracil. Procarbezine may be used in the dispensing device of the invention at a level of 2-4 mg for treating gliomas every 2 to 4 weeks by implantation.

Implants, according to the invention, may be used to deliver analgesic/antiinflammatory drugs such as indomethacin or other NSAIDs such as aspirin, naproxen, ibuprofen, and the like directly to the tissues surrounding joints. With the adverse event profiles of oral NSAID's and COX-2 inhibitors, this offers the potential of greater efficacy than oral treatments, while potentially reducing the side effects associated with circulating levels of these drugs. When a joint is treated with an anti-inflammatory drug such as triamcinolone, the dose may be 20 to 40 mg with or without 2-4 mg of dexamethasone in the hypercompressed microcapsules.

### EXAMPLE

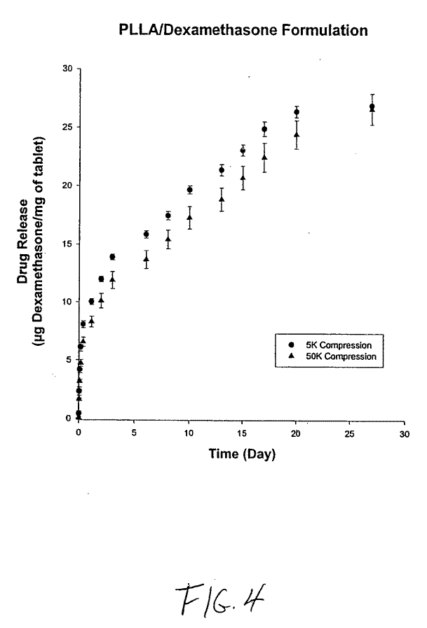

A dosage formulation of dexamethasone as a compressed microcapsule formulation is prepared by dispersing 325 mg of dexamethasone in 5 g of a poly(dl-lactide) polymer (PLA) (intrinsic viscosity 0.66-0.80 dl/g as measured in a Ubbelohde viscometer by assessing the flow time of polymer solutions; PLA is soluble in acetone, chloroform or dichloromethane) dissolved in 125 ml of chloroform and 3.5 ml of ethanol. The suspension is agitated between 1500 to 2000 RPM with 700 ml of a 2% polyvinyl alcohol (30K to 70K MW) maintained at 4° C. After 6 hours of stirring, the agitating speed is reduced to 700 RPM and chloroform is allowed to evaporate over night. The microspheres formed are recovered by centrifugation at 1500 RPM, washed 3 times with water and lyophilized. The microspheres form a free flowing powder having 6.5 wt % of dexamethasone with the microspheres having a general diameter in the range of 5 to 25 microns. Thereafter, 250 mg of the microspheres are placed in 7 mm diameter stainless steel mold (used for conventional tablet preparation in the pharmaceutical industry) in a MTS mechanical tester modified for compression. A compression force of 5K is used to form a first dispensing device and a pressure of 50K psi is used to form a second dispensing device using 60 mg of microspheres. The thickness of pellets formed by applying 5K psi of compression pressure is approximately 5.8 mm with a density of 1.06, whereas the thickness for the pellet prepared by applying 50K psi of pressure is approximately 4.2 mm with a density of about 1.55. The dosage form prepared by 50K psi contained 40% more material (by weight) than the dosage form prepared with 5K psi. The dispensing devices prepared using 5K psi and 50K psi were both placed in water. The disc made with 5K psi rapidly disintegrated and dispersed. When the disc made with 5K psi and the disc made with 50K psi were placed in pH 7.4 phosphate buffer, both discs rapidly disintegrated. The in vitro release of dexamethasone from both the 5K psi and 50K psi discs was measured over a 24 hour period of time by placing each disc in a container and filled with pH 7.4 PBS. The containers were placed on an orbital shaker (at ambient temperature) rotating at 100 RPM. At pre-determined time-points, samples were withdrawn and the containers were replenished with fresh aliquots of PBS and the amount of dexamethasone released was determined and is shown in FIG. 4. The results shows that the microspheres provide a very moderate initial burst release of dexamethasone which becomes a pseudo-first order release after one day. The 5K psi showed about a 20% faster release than the 50K psi disc during this test.

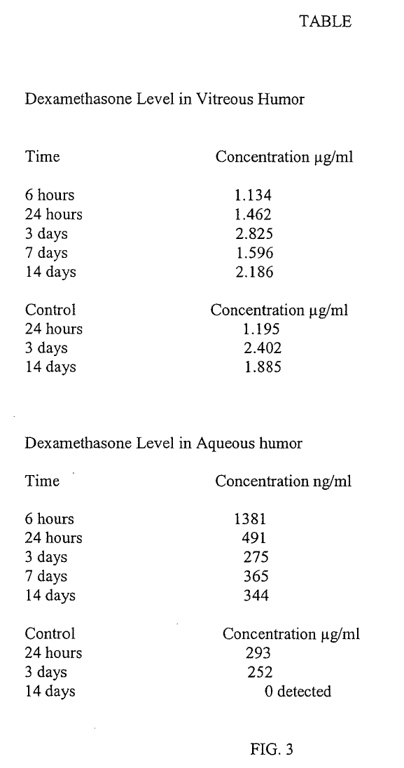

Discs measuring 7 mm in diameter, having a thickness of 1 mm, a weight of about 60 mg and a drug loading of 6.5% are made with 50K psi using dexamethasone and the polymer system described above. These discs are placed beneath the conjunctiva in the super temporal quadrant of the eyes of five pigs. The level of dexamethasone in the aqueous humor and the vitreous humor is determined at 0.25 day, 1 day, 3 days, 7 days and 14 days by sampling and analyzing the vitreous humor and the aqueous humor. The concentrations of dexamethasone are reported in FIG. 3. The release profile shown in FIG. 3 shows that the 50K psi disc provided sustained release of dexamethasone for the entire 14 days of the study. Tests of plasma found no detectable dexamethasone which confirmed that the controlled release dosage form has no systemic effect.

1. A dispensing device which comprises a polymer which is combined with a therapeutic agent in the form of micro or nano particles which are compressed to form a controlled release dispensing unit.

2. A dispensing device as defined in claim 1 where the therapeutic agent is selected from the group consisting of steroids, non-steroidal anti-inflammatory drugs, antihistamines, antibiotics, mydriatics, beta-adrenergic antagonists anesthetics, alpha-2-beta adrenergic agonists, mast cell stabilizers, prostaglandin analogues, sympathomimetics, parasympathomimetics, antiproliferative agents, agents to reduce ocular angiogenesis and neovascularization, vasoconstrictors, anti-neoplastic agents, a polynucleotide, or a recombinant protein analog an angiogenic inhibitors and combinations thereof.

3. A dispensing device as defined in claim 1 where the polymer is selected from the group consisting of poly(alpha hydroxy butyric acid), poly(p-dioxanone) poly(l-lactide), poly(dl-lactide), polyglycolide, poly(glycolide-co-lactide), poly(glycolide-co-dl-lactide), a block polymer of polyglycolide, trimethylene carbonate and polyethylene oxide, or a mixture of any of the foregoing.

4. A dispensing device as defined in claim 2 where the polymer is biodegradable.

5. A dispensing device as defined in claim 4 where the microcapsule which has been compressed by the application of 12,000 to 200,000 psi.

6. A dispensing device as defined in claim 4 where the microcapsule which has been compressed by the application of 25,000 to 50,000 psi.

7. A dispensing device as defined in claim 4 where the microcapsule which has been compressed by the application of 50,000 psi.

8. A dispensing device as defined in claim 7 where the therapeutic agent is a steroid.

9. A method of locally administering a drug which comprises forming a dispensing device comprising a polymer in combination with a therapeutic agent in the form of a microparticle which is compressed to form a controlled release dispensing unit and thereafter placing said dispensing unit in a patient in a location that will provide for release of the drug.

10. A method as defined in claim 9 where the therapeutic agent is selected from the group consisting of: steroids, non-steroidal anti-inflammatory drugs, antihistamines, antibiotics, mydriatics, beta-adrenergic antagonists, anesthetics, alpha-2-beta adrenergic agonists, mast cell stabilizers, prostaglandin analogues, sympathomimetics, parasympathomimetics, antiproliferative agents, agents to reduce ocular angiogenesis and neovascularization, vasoconstrictors and combinations thereof.

11. A method as defined in claim 10 where the polymer is selected from the group consisting of poly(alpha hydroxy butyric acid), poly(p-dioxanone) poly(l-lactide), poly(dl-lactide), polyglycolide, poly(glycolide-co-lactide), poly(glycolide-co-dl-lactide), a block polymer of polyglycolide, trimethylene carbonate and polyethylene oxide, or a mixture of any of the foregoing.

12. A method as defined in claim 10 where the polymer and the therapeutic agent are in the form of a rod.

13. A method as defined in claim 12 where the microparticles have been compressed by the application of 12,000 to 200,000 psi.

14. A method as defined in claim 12 where the microparticles have been compressed by the application of 25,000 to 50,000 psi.

15. A method as defined in claim 12 where the therapeutic agent is a steroid.

16. A method as defined in claim 14 where the microparticles have been compressed by the application of 50,000 psi.

17. A method of locally administering a drug which comprises forming a dispensing device comprising a polymer in combination with a therapeutic agent in the form of a microparticle which is compressed to form a controlled release dispensing unit and thereafter placing said dispensing unit in contact with an injectable liquid to disperse the microparticles and form a suspension of microparticles prior to placing said suspension in a patient in a location that will provide for release of the drug.
